(19) United States
(12) Patent Application Publication
(10) Pub. No.: US 2004/0034382 A1
(43) Pub. Date: Feb. 19, 2004

(54) Trephination tool

(76) Inventor: Jeffrey E. Thomas, Albuquerque, NM (US)

Correspondence Address: BENESCH, FRIEDLANDER, COPLAN & ARONOFF LLP, ATTN: IP DEPARTMENT DOCKET CLERK, 2300 BP TOWER, 200 PUBLIC SQUARE, CLEVELAND, OH, 44114, US

(21) Appl. No.: 10/217,552

(22) Filed: Aug. 13, 2002

(51) Int. Cl. A61B 17/32

(52) U.S. Cl. 606/180; 606/172

(57) ABSTRACT

A device perpendicularly aligns an axis to a tangent plane at a point on a nonplanar surface. The device includes an annular cutting surface and a port. The annular cutting surface is centered on the axis and rotatable about the axis. The port is centered on the axis. An insertion instrument inserted through the port is perpendicular to the tangent plane when the annular cutting surface cuts into the nonplanar surface.

### FIELD OF THE INVENTION

The invention generally relates to trephination instruments. The invention finds particular application in medical devices for penetrating the skull.

### BACKGROUND OF THE INVENTION

Most surgical neurological procedures require accurate placement of medical instruments within the patient's brain. A common neurological surgical procedure is a ventriculostomy in which a cerebral ventricle drain, shunt or catheter is implanted. Procedures for the placement of ventricular drains, shunts and catheters rely on the skill of the neurosurgeon. After imaging the brain, the neurosurgeon forms a burr hole in the skull and guides a catheter through the burr hole toward landmarks on the ipsilateral or contralateral side of the patient's head by assuming a path perpendicular to the surface of the head.

Neurosurgeons attempt to perforate a patient's cranium at an angle 90° to a tangent plane to the surface (i.e., perpendicular to the surface). The perpendicular orientation may prevent injury and also may locate the catheter more precisely with respect to the landmarks. For example, a burr hole drilled at an orientation of 90° to the surface of the skull may prevent injury to the underlying dura and brain tissue that may otherwise be caused due to the continued rotation of the metal bit of the drilling apparatus once the bit tip exits the skull. The properly drilled hole may also ensure that a ventricular catheter subsequently inserted into the brain, perpendicular to the curvature of the cranium, will not deviate from its intended path due to a misaligned skull hole.

### SUMMARY

An aspect of the invention provides a device for perpendicularly aligning an axis to a tangent plane at a point on a nonplanar surface. The device includes an annular cutting surface and a port. The annular cutting surface is centered on the axis and rotatable about the axis. The port is centered on the axis. An insertion instrument inserted through the port is perpendicular to the tangent plane when the annular cutting surface cuts into the nonplanar surface.

Another aspect of the invention provides a method of perpendicularly aligning a port to a tangent plane at a point on a surface. The method centers an annular cutting surface on an axis extending through the port and the point on the surface. The annular cutting surface rotates such that the annular cutting surface cuts into the surface. The annular cutting surface perpendicularly aligns the port with the tangent plane.

### BRIEF DESCRIPTION OF THE DRAWINGS

FIG. 1 is a perspective view of a trephination device suitable to practice the present invention;

FIG. 2 is a top plan view of the device of FIG. 1;

FIG. 3 is a cross sectional view of the device of FIG. 1;

FIG. 4 is a perspective view of another embodiment suitable to practice the present invention; and

FIG. 5 is a cross sectional view of another embodiment suitable to practice the present invention.

### DETAILED DESCRIPTION OF THE PREFERRED EMBODIMENTS

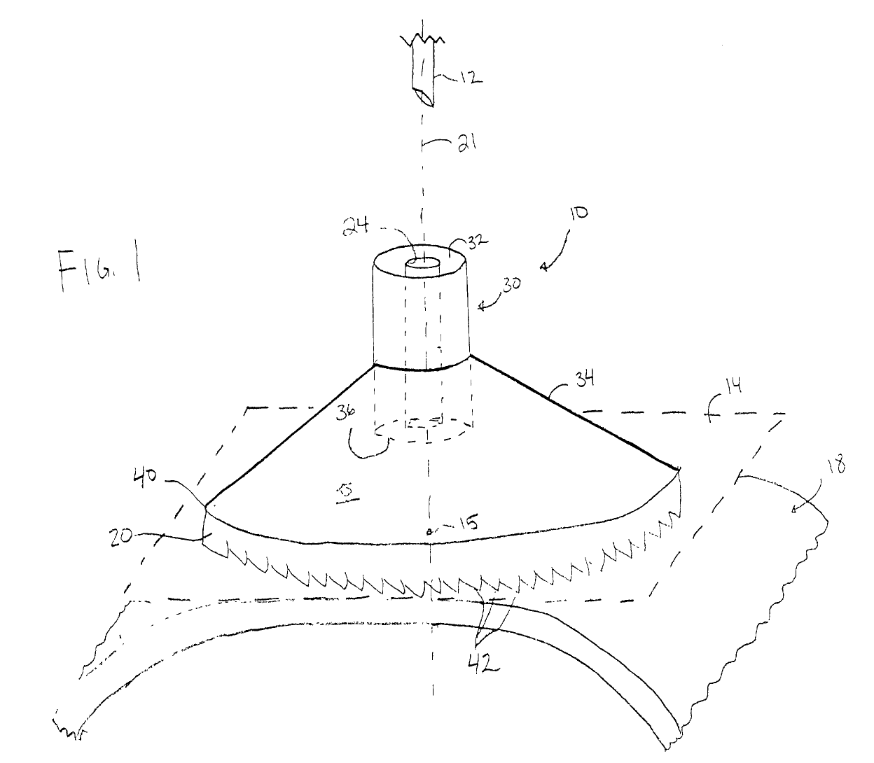

With respect to the drawing figures, FIG. 1 is a perspective view of a trephination device 10 suitable to practice the present invention. The device 10 perpendicularly aligns an insertion instrument 12 to a tangent plane 14 at a point 16 on a nonplanar surface 18. An annular cutting surface 20 is centered on an axis 21 extending through the point 16 on the nonplanar surface 18. A port 24 is centered on the axis 21 and configured to receive the insertion instrument 12. The insertion instrument 12 is inserted through the port 24 perpendicular to the tangent plane 14 when the annular cutting surface 20 cuts into the nonplanar surface 18. For example, the device 10 may be used for directing a catheter perpendicular into a skull cavity of a patient by perpendicularly aligning the port 24 with a point on the outer surface of the skull.

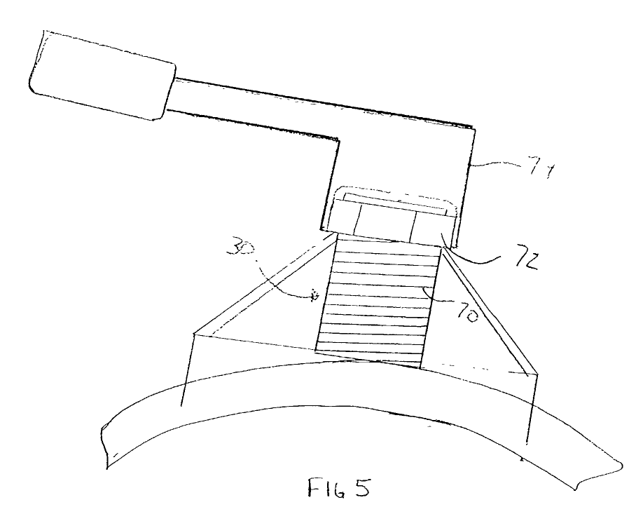

The device 10 includes a cylindrical column 30 centered on the axis 21. A top end 32 of the column 30 is configured to couple the device 10 to a torque producing device such as a torque wrench or a drill (either a hand or power drill, FIG. 5). A frustro-conical wall 34 is coupled to the column 30. The wall 34 is also centered on the axis 21. The wall 34 encloses a recess 35 within the device 10. A bottom end 36 of the column 30 extends into the recess 34. The annular cutting surface 20 extends circumferentially around a bottom end 40 of the wall 34. Teeth 42 of the annular cutting surface 20 extend in a direction parallel to the axis 21 and centered on the axis 21.

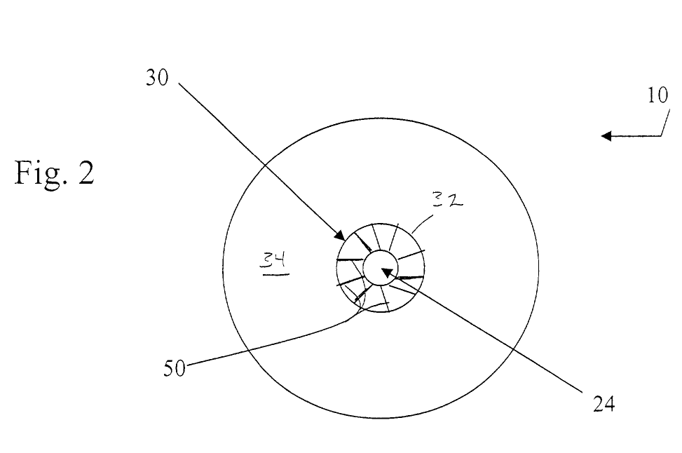

With respect to FIG. 2, a top plan view of the device 10 of FIG. 1 is shown. The top end 32 of the column 30 includes ridges 50 that are configured to mate with the torque producing device. Those skilled in the art will appreciate that alternate methods of coupling to the torque producing device, such as a cap removably engagable with a threaded top end 32, or other such arrangements can be substituted with no loss of functionality. The coupling allows a user to generate torque in the device 10 by allowing a longer moment arm to attach to the device 10 along the axis 21 and rotate the device 10 relative to the nonplanar surface 18.

The port 24 allows access to the recess 35 so that the insertion instrument 12 advanced through the port 24 may also advance through the recess 35 to the nonplanar surface 18. The column 30 is elongated so that the insertion instrument 12 can remain perpendicular to the annular cutting surface 20 and perpendicular to the tangent plane 14. The port 24 has a radius sized to pass the insertion instrument 12 through the port 24. For example, the port 24 may be sized to pass a catheter through the device 10. By maintaining a radius similar in size to the insertion instrument 12, the port 24 can contribute to maintaining the perpendicular orientation of the insertion instrument 12 to the tangent plane 14.

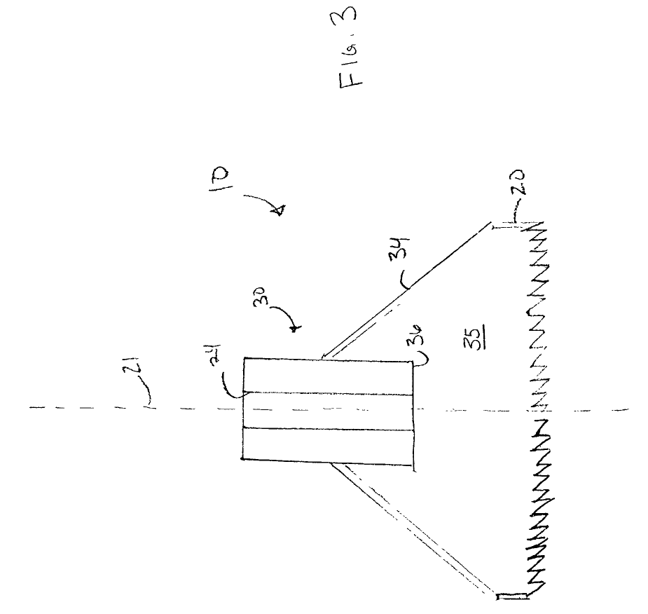

With respect to FIG. 3, a cutaway view of the device of FIG. 1 is shown. The column 30 extends into the recess 35. The positioning of the column 30 within recess 35 acts as a depth gauge for the device 10. When the annular cutting surface 20 is cutting through the nonplanar surface 18, the bottom end 32 of the column 30 prevents the device 10 from proceeding past a desired depth in the nonplanar surface 18 by acting as a mechanical stop. As the annular cutting surface 20 cuts through the nonplanar surface 18, the bottom end 32 of column 30 abuts the nonplanar surface 18 and stops the device 10 from cutting any deeper into the nonplanar surface 18. For example, when the device 10 is used to trephinate the skull, the column 30 can be extended into the recess 35 to a depth so that the distance from the annular cutting surface to the bottom end 36 of the column is equal to the distance from the nonplanar surface 18 to the inner table of the skull. In this manner, the device 10 desirably minimizes the likelihood of cutting into the dura of the brain or alternatively may be stopped prior to cutting through the skull.

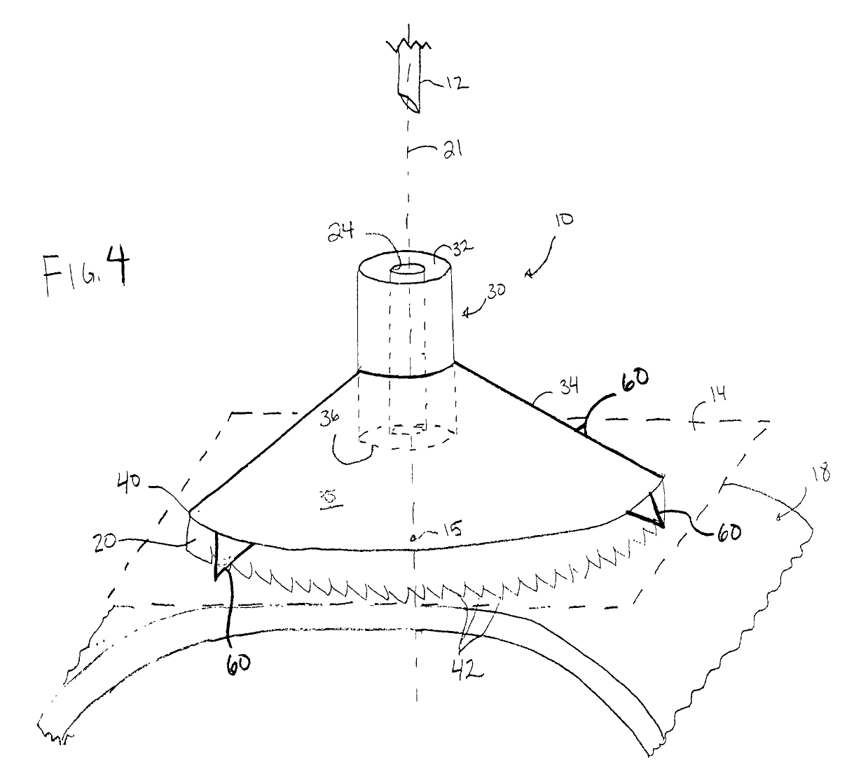

With respect to FIG. 4, a perspective view of another embodiment of the present invention is shown. A set of guides 60 are coupled to the annular cutting surface 20. The guides 60 are configured so that when the device 10 drills into the nonplanar surface 18, the guides 60 may advance to the nonplanar surface 18. The guides 60 may be adjustable so that the depth of the cut can be controlled. For example, the annular cutting surface 20 may be configured with external threads and the guides 60 may be attached to each other with a ring so that the guides 60 may rotate relative to the device 10. Alternatively, the guides 60 may ride in slots extending vertically along the annular cutting surface 20. The guides 60 can be advanced in the slots and locked into place by a spring mechanism. By setting the vertical distance between the bottom end 40 of the wall 34 to the guides 60, a user desirably limits the depth the annular cutting surface 20 cuts into the nonplanar surface 18. Those skilled in the art will appreciate that alternate methods of coupling the guides 60 to the annular cutting surface 20 can be substituted with no loss of functionality.

As shown in the embodiment of FIG. 5, the column 30 may have external threads 70 so that the depth of the column 30 can be adjusted. A lock washer 72 may be used to lock the column in place with respect to the wall 34 when the column 30 is adjusted to its proper depth. A torque producing device 74 couples to the column 30 at the lock washer 72 and rotates the device 10 with respect to the nonplanar surface 18.

In operation, the device 10 is used to perpendicularly align an insertion instrument to the tangent plane 14 at the point 15 on the nonplanar surface 18. The device 10 is centered on the axis 21 so that the axis 21 extends through the point 15 on the nonplanar surface 18. The device 10 is rotated relative to the nonplanar surface 18 so that the annular cutting surface 20 cuts into the nonplanar surface 18. The device 10 advances into the nonplanar surface 18 to a depth prescribed by the depth the column 30 is advanced into the recess 35. In operation, the depth of column 30 may be adjusted to limit the depth the torque producing device 74 drills into the nonplanar surface 18.

Once the annular cutting surface 20 has cut into the nonplanar surface 18, then the port 21 is perpendicularly aligned with the tangent plane 14. A drill then drills through the port 24 and through the point 15 on the nonplanar surface 18. The recess 35 may house any chips or fluid that are released during the drilling process. The drill advances to penetrate the nonplanar surface 18. When the drill has penetrated the nonplanar surface 18, then the drill is removed from the port 21.

The insertion instrument 12 is advanced through the port 24 through the drilled hole and into a cavity under the nonplanar surface 18. For example, the device 10 may be used to perpendicularly align the port 24 to a point on a skull. The insertion instrument 12 is inserted through the port 24 centered on the axis 21. The insertion instrument 12 then is inserted through the drilled hole through the skull and into the skull cavity.

The device 10 may be included in a kit for treating stroke patients. The kit may include the device 10, medication, and other tools to access the cerebrospinal fluid. The tools may include surgical instruments such as shaving instruments, a dural incision blade, sutures and dressings. The surgical instruments may prepare the patient's head by being used for shaving off hair, sterilizing the point 15, and dressing the head after the surgery. Insertion instruments may also be included in the kit. For example, the kit may include needles, tunnelling devices and catheters designed for reaching the ventricles, subarachnoid space, and lumbar. The device 10 can be quickly used to map a perpendicular path between the point on the surface of the skull and the affected area of the brain. Because the device creates a perpendicular distance to the affected area, the amount of damage to the brain may be minimized because the shortest distance is taken. Additionally, a surgeon may not need to realign his entry and, thus, make fewer mistakes in directing the tools to the proper site.

There have been described and illustrated herein embodiments of the apparatus and method of the present invention. While in accordance with the patent statutes, a preferred embodiment has been presented, it is not intended that the scope of the invention be limited thereto. It is intended that the invention be as broad in scope as the art will allow and that the specification be read likewise. For example, those skilled in the art will appreciate that certain features of one embodiment may be combined with features of another embodiment to provide yet additional embodiments. It will, therefore, be appreciated by those skilled in the art that other modifications could be made to the provided invention without deviating from its spirit and scope as so claimed and described.

1. A device for perpendicularly aligning an axis to a tangent plane at a point on a nonplanar surface, comprising:
an annular cutting surface centered on the axis and rotatable about the axis; and
a port centered on the axis such that an insertion instrument inserted through the port is perpendicular to the tangent plane when the annular cutting surface cuts into the nonplanar surface.

2. The device of claim 1, wherein the nonplanar surface is the outer table of a skull.

3. The device of claim 2, wherein the annular cutting surface is configured to cut from the outer table of the skull to the inner table of the skull.

4. The device of claim 1, further comprising a depth gauge configured to limit the depth the annular cutting surface cuts through the nonplanar surface.

5. The device of claim 4, wherein the depth gauge includes a mechanical stop that abuts the nonplanar surface when the annular cutting surface cuts through the nonplanar surface.

6. The device of claim 1, further comprising a mating mechanism configured to mate the device to a torque generating tool.

7. The device of claim 6, wherein the torque generating tool is a hand drill.

8. A method of perpendicularly aligning a port to a tangent plane at a point on a surface, comprising the steps of:
centering an annular cutting surface on an axis extending through the port and the point on the surface; and
rotating the annular cutting surface such that the annular cutting surface cuts into the surface and perpendicularly aligns the port with the tangent plane.

9. The method of claim 8, further comprising the step of drilling a hole through the surface along the axis.

10. The method of claim 9, further comprising the step of limiting the depth of the hole by adjusting the distance between the port and the surface.

11. The method of claim 9, further comprising the step of guiding an insertion instrument through the port and through the hole.

12. The method of claim 8, wherein the surface is the outer table of a skull.

13. The method of claim 12, wherein the annular cutting surface is configured to cut from the outer table of the skull to the inner table of the skull.

14. The method of claim 8, further comprising the step of setting a depth gauge configured to limit the depth the annular cutting surface cuts through the surface.

15. The method of claim 14, wherein the depth gauge is a mechanical stop that abuts the surface when the annular cutting surface cuts through the surface.

16. The method of claim 8, further comprising the step of mating a torque generating tool to the port such that the torque generating tool rotates the annular cutting surface.

17. The method of claim 16, wherein the torque generating tool is a hand drill.

18. A kit for delivering medication to a patient's brain, comprising:
an insertion instrument configured to deliver medication to a portion of the brain intersected by an axis extending from the skull through the brain; and
a device for perpendicularly aligning the axis to a tangent plane at a point on the skull, comprising:
an annular cutting surface centered on the axis and rotatable about the axis; and
a port centered on the axis such that the insertion instrument inserted through the port is perpendicular to the tangent plane when the annular cutting surface cuts into the nonplanar surface.

19. The kit of claim 18, wherein the portion of the brain is a ventricle.

20. The kit of claim 18, further comprising surgical instruments configured to prepare the skull for the annular cutting surface.
